(19) United States
(12) Patent Application Publication
(10) Pub. No.: US 2026/0137740 A1
(43) Pub. Date: May 21, 2026

(54) IRF MODULATOR-EXPRESSING ONCOLYTIC VIRUSES FOR TREATING CANCER

(71) Applicant: UNIVERSITY OF PITTSBURGH - OF THE COMMONWEALTH SYSTEM OF HIGHER EDUCATION, Pittsburgh, PA (US)

(72) Inventors: Saumendra N. SARKAR, Wexford, PA (US); Stephen Howard THORNE, Pittsburgh, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 19/410,748

(22) Filed: Dec. 5, 2025

Related U.S. Application Data
(63) Continuation of application No. 17/869,943, filed on Jul. 21, 2022, now abandoned.
Application No. 17/869,943 is a continuation of application No. PCT/US2021/021173, filed on Mar. 5, 2021.
(60) Provisional application No. 62/985,979, filed on Mar. 6, 2020.

(51) Int. Cl. A61K 35/768; A61K 39/395; A61P 35/00; C07K 14/47; C12N 15/86

(52) U.S. Cl. CPC A61K 35/768; A61K 39/3955; A61P 35/00; C07K 14/4702; C12N 15/86; C12N 2710/24132; C12N 2710/24143

(57) ABSTRACT

The present disclosure provides oncolytic viruses expressing a modulator of interferon regulatory factors (IRFs), and compositions comprising thereof. The present disclosure further provides methods of using said oncolytic viruses and compositions for treating cancer, and for improving a subject's responsiveness to an immunomodulatory agent (e.g., an immune checkpoint inhibitor).

### CROSS-REFERENCE TO RELATED APPLICATIONS

This application is a continuation of U.S. patent application Ser. No. 17/869,943, filed Jul. 21, 2022, which is a continuation of International Patent Application No. PCT/US2021/021173, filed Mar. 5, 2021, which claims priority to U.S. Provisional Patent Application Ser. No. 62/985,979, filed on Mar. 6, 2020, the contents of which are hereby incorporated by reference in their entireties, and to each of which priority is claimed.

### GRANT INFORMATION

This invention was made with government support under grant number CA178766 awarded by the National Institutes of Health. The government has certain rights in the invention.

### SEQUENCE LISTINGS

The instant application contains a Sequence Listing, which has been submitted in XML format via EFS-Web and is hereby incorporated by reference in its entirety. Said XML copy, created on Dec. 3, 2025, is named 072396_1132_ST26.xml and is 3,561 bytes in size.

### 1. TECHNICAL FIELD

The present disclosure provides oncolytic viruses expressing a modulator of interferon regulatory factors (IRFs) (i.e., an IRF modulator), and compositions comprising thereof. The present disclosure further provides methods of using said oncolytic viruses and compositions for treating cancer, and for improving a subject's responsiveness to an immunomodulatory agent (e.g., an immune checkpoint inhibitor).

### 2. BACKGROUND

Immunotherapies such as immune checkpoint inhibitors (e.g., anti-PD-1 antibodies or anti-CTLA-4 antibodies) have entered the mainstream of cancer treatment. However, these therapies as single modality treatments or even in combination with each other only benefit a subset of patients. There is growing literature showing that patients who had previously responded to immune checkpoint inhibitors can develop resistance to the immune checkpoint inhibitors later.

Oncolytic virus (OV)-based cancer therapy is a form of immunotherapy that employs viruses that can selectively infect and lyse tumor cells, while exerting minimal or no pathogenicity against normal non-neoplastic host cells. Besides the direct killing (oncolysis) ability, oncolytic viruses can also induce anti-tumoral immune responses of the host. However, OV-based cancer therapy has limited effectiveness in clinical applications.

Thus, there remain needs for methods and compositions for improving cancer patients' responsiveness to immunotherapies (e.g., immune checkpoint inhibitors), and for improving the efficacy of OV-based cancer therapy.

### 3. SUMMARY OF THE INVENTION

The present disclosure provides oncolytic viruses expressing a modulator of interferon regulatory factors (IRFs), and compositions comprising thereof. It is based, at least in part, on the discovery that delivering an IRF1 inhibitor-expressing oncolytic virus to tumors inhibited the growth of tumors in vivo.

In one aspect, the present disclosure provides an oncolytic virus comprising a nucleic acid molecule that encodes a modulator of an interferon regulatory factor (IRF).

In certain embodiments, the IRF is IRF1, IRF3, IRF7, or a combination thereof. In certain embodiments, the IRF is IRF1. In certain embodiments, the modulator inhibits the activity of the IRF. In certain embodiments, the modulator inhibits the activity of IRF1.

In certain embodiments, the modulator is IRF2. In certain embodiments, the IRF2 is a human IRF2 or a mouse IRF2.

In certain embodiments, the modulator reduces IRF-mediated gene expression. In certain embodiments, the modulator reduces the expression of CD274 gene.

In certain embodiments, the nucleic acid molecule is an exogenous nucleic acid molecule. In certain embodiments, the nucleic acid molecule is integrated into the genome of the oncolytic virus.

In certain embodiments, the oncolytic virus is an oncolytic vaccinia virus. In certain embodiments, the oncolytic vaccinia virus lacks the expression of a functional thymidine kinase (TK).

In another aspect, the present disclosure provides a method of treating a subject having cancer, comprising administering to the subject a presently disclosed oncolytic virus. In certain embodiments, the subject is a human subject.

In certain embodiments, the presently disclosed method further comprises administering an immunomodulatory agent to the subject. In certain embodiments, the immunomodulatory agent is selected from the group consisting of immune checkpoint inhibitors, T cells, dendritic cells, therapeutic antibodies, cancer vaccines, cytokines, Bacillus Calmette-Guérin (BCG), and any combinations thereof. In certain embodiments, the immunomodulatory agent is an immune checkpoint inhibitor. In certain embodiments, the immune checkpoint inhibitor is selected from the group consisting of anti-PD1 antibodies, anti-PD-L1 antibodies, anti-CTLA-4 antibodies, anti-BTLA antibodies, anti-TIM3 antibodies, anti-LAG-3 antibodies, and any combinations thereof. In certain embodiments, the immune checkpoint inhibitor is an anti-PD-L1 antibody or an anti-CTLA-4 antibody.

In certain embodiments, the cancer is a solid tumor. In certain embodiments, the cancer is selected from the group consisting of adenocarcinomas, osteosarcomas, cervical carcinomas, melanomas, hepatocellular carcinomas, breast cancers, lung cancers, prostate cancers, ovarian cancers, leukemias, lymphomas, renal carcinomas, pancreatic cancers, gastric cancers, colon cancers, duodenal cancers, glioblastoma multiforme, astrocytomas, sarcomas, and combinations thereof. In certain embodiments, the cancer is melanoma or renal carcinoma.

In another aspect, the present disclosure provides a method for improving a subject's responsiveness to an immunomodulatory agent, comprising administering to the subject a presently disclosed oncolytic virus, wherein the subject has cancer. In certain embodiments, the subject is a human subject.

In certain embodiments, the subject was previously treated with the immunomodulatory agent. In certain embodiments, the subject has developed a resistance to the immunomodulatory agent. In certain embodiments, the presently disclosed method further comprises administering an immunomodulatory agent to the subject. In certain embodiments, the immunomodulatory agent is selected from the group consisting of immune checkpoint inhibitors, T cells, dendritic cells, therapeutic antibodies, cancer vaccines, cytokines, Bacillus Calmette-Guérin (BCG), and any combinations thereof. In certain embodiments, the immunomodulatory agent is an immune checkpoint inhibitor. In certain embodiments, the immune checkpoint inhibitor is selected from the group consisting of anti-PD1 antibodies, anti-PD-L1 antibodies, anti-CTLA-4 antibodies, anti-BTLA antibodies, anti-TIM3 antibodies, anti-LAG-3 antibodies, and any combinations thereof. In certain embodiments, the immune checkpoint inhibitor is an anti-PD-L1 antibody or an anti-CTLA-4 antibody.

In certain embodiments, the cancer is a solid tumor. In certain embodiments, the cancer is selected from the group consisting of adenocarcinomas, osteosarcomas, cervical carcinomas, melanomas, hepatocellular carcinomas, breast cancers, lung cancers, prostate cancers, ovarian cancers, leukemias, lymphomas, renal carcinomas, pancreatic cancers, gastric cancers, colon cancers, duodenal cancers, glioblastoma multiforme, astrocytomas, sarcomas, and combinations thereof. In certain embodiments, the cancer is melanoma or renal carcinoma.

In another aspect, the present disclosure provides a pharmaceutical composition comprising a presently disclosed oncolytic virus.

In certain embodiments, the presently disclosed pharmaceutical composition further comprises an immunomodulatory agent. In certain embodiments, the immunomodulatory agent is selected from the group consisting of immune checkpoint inhibitors, T cells, dendritic cells, therapeutic antibodies, cancer vaccines, cytokines, Bacillus Calmette-Guérin (BCG), and any combinations thereof. In certain embodiments, the immunomodulatory agent is an immune checkpoint inhibitor. In certain embodiments, the immune checkpoint inhibitor is selected from the group consisting of anti-PD1 antibodies, anti-PD-L1 antibodies, anti-CTLA-4 antibodies, anti-BTLA antibodies, anti-TIM3 antibodies, anti-LAG-3 antibodies, and any combinations thereof. In certain embodiments, the immune checkpoint inhibitor is an anti-PD-L1 antibody or an anti-CTLA-4 antibody.

In certain embodiments, the presently disclosed pharmaceutical compositions further comprise a pharmaceutically acceptable carrier.

In certain embodiments, the presently disclosed pharmaceutical compositions are for treating a subject having cancer or improving a subject's responsiveness to an immunomodulatory agent.

In another aspect, the present disclosure provides a kit comprising a presently disclosed oncolytic virus, or a presently disclosed pharmaceutical composition. In certain embodiments, the presently disclosed kit further comprises an immunomodulatory agent. In certain embodiments, the immunomodulatory agent is an immune checkpoint inhibitor. In certain embodiments, the immune checkpoint inhibitor is selected from the group consisting of anti-PD1 antibodies, anti-PD-L1 antibodies, anti-CTLA-4 antibodies, anti-BTLA antibodies, anti-TIM3 antibodies, anti-LAG-3 antibodies, and any combinations thereof. In certain embodiments, the immune checkpoint inhibitor is an anti-PD-L1 antibody or an anti-CTLA-4 antibody.

In certain embodiments, the kit further comprises instructions for treating a subject having cancer or improving a subject's responsiveness to an immunomodulatory agent.

### 4. BRIEF DESCRIPTION OF THE DRAWINGS

FIGS. 1A-1D show that IRF2 promoted tumor regression. FIGS. 1A-1B provides quantitation of PD-L1 expression by flow cytometry in human MEL-285 melanoma cells (FIG. 1A) and mouse B16 melanoma cells (FIG. 1B). Human MEL-285 melanoma cells were transfected with human IRF2-expressing vectors or control vectors, followed by IFN-γ stimulation. Mouse B16 melanoma cells were transfected with mouse Irf2 (mIrf2)-expressing vectors or control vectors, followed by IFN-γ stimulation. FIG. 1C provides tumor volumes measured from day 0 to day 22 in mice implanted with mouse B16 melanoma cells, and received oncolytic vaccinia virus carrying mouse Irf2 (VV-mIrf2) or control vaccinia virus (VV-control) treatments. FIG. 1D provides tumor volumes measured in BALB/C mice injected with RENCA tumors followed by VV-mIrf2 or VV-control treatments.

### 5. DETAILED DESCRIPTION

Non-limiting embodiments of the present disclosure are described by the present specification and Examples. For purposes of clarity of disclosure and not by way of limitation, the detailed description is divided into the following subsections:
5.1. Definitions;
5.2. Oncolytic Viruses Expressing an IRF Modulator;
5.3. Pharmaceutical Compositions;
5.4. Methods of Treatment; and
5.5. Kits.

### 5.1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “modulator” as used herein as a “modulator of an interferon regulatory factor (IRF)” or interchangeably an “IRF modulator” refers to a molecule that can regulate the activity of an IRF. In certain embodiments, the modulator can inhibit the activity of an IRF. In certain embodiments, the modulator is a protein molecule (e.g., IRF2).

As used herein, the term “oncolytic virus” or “OV” refers to a virus capable of selectively replicating in a cancer cell, and slowing the growth or inducing the death of the cancer cell, either in vitro or in vivo, while having no or minimal effect on normal cells. In certain embodiments, the oncolytic viruses spread within a tumor without causing damages to non-cancerous tissues. In certain embodiments, the oncolytic viruses do not replicate or replicate at a reduced speed in non-cancer cells as compared to in cancer cells. Non-limiting exemplary oncolytic viruses include Coxsackieviruses, Maraba viruses (rhabdovirus), Parvoviruses, Seneca Valley viruses, vesicular stomatitis viruses (VSVs), Newcastle disease viruses (NDVs), retroviruses, reoviruses, measles viruses, Sindbis viruses, influenza viruses, herpes simplex viruses (HSVs), Sendai viruses, vaccinia viruses (VVs), and adenoviruses, and variants thereof.

As used herein, the term “vaccinia virus” or “VV” refers to an enveloped DNA virus from the poxvirus family. In certain embodiments, the VV comprises a linear, double-stranded DNA genome of about 200 kb. Non-limiting examples of vaccinia virus strains include strains of, derived from, or modified forms of Western Reserve (WR) strain, Tashkent strain, Lister strain (also known as Elstree), Dryvax strain (also known as Wyeth strain), IHD-J strain, and IHD-W strain, Brighton strain, Ankara strain, modified vaccinia Ankara (MVA) strain, Dairen strains (e.g., Dairen I strain (DIs)), LIPV strain, lister clone 16m8 (LC16m8) strain, LC16MO strain, LIVP strain, WR 65-16 strain, Connaught strain, New York City Board of Health (NYCBH) strain, EM63 strain, ACAM2000™ strain, CV-1 strain, Paris strain, Copenhagen (Cop) strain, Bern strain, and the Tian Tan (VTT) strain.

As used herein, the term “mutation” refers to a mutation in an amino acid sequence or in a nucleotide sequence. In certain embodiments, a mutation in an amino acid sequence can be a substitution (replacement), an insertion (addition), or a deletion (truncation) of at least one amino acid in the amino acid sequence. In certain embodiments, a mutation in a nucleotide sequence can be a substitution (replacement), an insertion (addition), or a deletion (truncation) of at least nucleotide of the nucleotide sequence.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “therapeutically effective amount” or “effective amount” refers to an amount of an oncolytic virus composition that is sufficient to reduce, inhibit, or abrogate tumor cell growth, in vitro or in vivo. In certain embodiments, the reduction, inhibition, or abrogation of tumor cell growth may be the result of necrosis, apoptosis, or an immune response. The amount of an oncolytic virus composition that is therapeutically effective or effective may vary depending on the context. An effective amount can be administered in one or more administrations.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more sign or symptoms, diminishment of extent of disease, stabilized (i.e., not worsening) state of disease, prevention of disease, delay or slowing of disease progression, and/or amelioration or palliation of the disease state. The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

### 5.2. Oncolytic Viruses Expressing an IRF Modulator

The present disclosure provides oncolytic viruses expressing a modulator of IRF (i.e., an IRF modulator). In certain embodiments, the oncolytic virus includes a nucleic acid molecule encoding the IRF modulator. In certain embodiments, the nucleic acid molecule is an exogenous nucleic acid molecule. In certain embodiments, the nucleic acid molecule is integrated into the genome of the oncolytic virus. The nucleic acid molecule encoding the IRF modulator can be a DNA molecule, an RNA molecule or a cDNA molecule to conform to the nucleic acid of the oncolytic viral genome into which it is integrated.

Interferon regulatory factors (IRFs) are a family of transcription factors that can regulate the expression of proteins involved in innate and adaptive immunities. There are currently nine IRFs in mammalians, including IRF1, IRF2, IRF3, IRF4 (i.e., PIP, ICSAT), IRF5, IRF6, IRF7, IRF8 (i.e., ICSBP) and IRF9 (i.e., p48, ISGF3γ). The present disclosure discovered that administering an agent (e.g., an IRF modulator) that inhibits the activity of IRFs in tumors in vivo can promote the anti-tumor immune response and inhibit the growth of tumors. The present disclosure further discovered that IRF-inhibiting agents can reduce the expression of programmed death-ligand 1 (PD-L1). PD-L1 plays an essential role in physiological immune homeostasis and is involved in the immune evasion activity employed by cancer cells. Reducing the expression of PD-L1 can improve the host anti-tumor immune response, and increase cancer cells' responsiveness to immunotherapies.

In certain embodiments, the presently disclosed oncolytic viruses express an IRF modulator that modulates the activity of an IRF, where the IRF suppresses the anti-tumor immunity. IRFs that can suppress the anti-tumor immunity include, but not limited to, IRF1, IRF3, and IRF7.

In certain embodiments, the IRF modulator (e.g., IRF2) inhibits (e.g., reduces or eliminates) the activity of IRF1, IRF3, IRF7, or a combination thereof. In certain embodiments, the IRF modulator inhibits the activity of IRF1. In certain embodiments, the IRF modulator inhibits (e.g., reduces or eliminates) the expression of genes regulated by IRF1. In certain embodiments, the IRF modulator inhibits, reduces, and/or eliminates the expression of CD274 gene (encoding PD-L1), ITGA8 gene, ENAH gene, PMP22 gene, SULF2 gene, CIITA gene, PGF gene COL4A1 gene, ERAPI gene, NNMT gene, AXL gene, or a combination thereof. In certain embodiments, the IRF modulator inhibits, reduces, and/or eliminates the levels of proteins expressed by CD274 gene, ITGA8 gene, ENAH gene, PMP22 gene, SULF2 gene, CIITA gene, PGF gene COL4A1 gene, ERAPI gene, NNMT gene, AXL gene, or a combination thereof. In certain embodiments, the IRF modulator reduces the expression of CD274 gene. In certain embodiments, the IRF modulator reduces the level of PD-L1 protein.

In certain embodiments, the IRF modulator is IRF2. IRF2 can competitively inhibit the IRF-mediated (e.g., IRF1-mediated) transcriptional activation of interferons alpha and beta, and other genes that employ IRF for transcription activation. In certain embodiments, the presently disclosed oncolytic viruses include a nucleic acid molecule that encodes IRF2.

In certain embodiments, the nucleic acid molecule encodes a human IRF2. In certain embodiments, the nucleic acid molecule encodes a human IRF2 having the amino acid sequence set forth in SEQ ID NO: 1.

[SEQ ID NO: 1]
MPVERMRMRPWLEEQINSNTIPGLKWLNKEKKIFQIPWMHAARHGWDV
EKDAPLFRNWAIHTGKHQPGVDKPDPKTWKANFRCAMNSLPDIEEVKDKS
IKKGNNAFRVYRMLPLSERPSKKGKKPKTEKEDKVKHIKQEPVESSLGLS
NGVSDLSPEYAVLTSTIKNEVDSTVNIIVVGQSHLDSNIENQEIVTNPPD
ICQVVEVTTESDEQPVSMSELYPLQISPVSSYAESETTDSVPSDEESAEG
RPHWRKRNIEGKQYLSNMGTRGSYLLPGMASFVTSNKPDLQVTIKEESNP
VPYNSSWPPFQDLPLSSSMTPASSSSRPDRETRASVIKKTSDITQARVKS
C

In certain embodiments, the human IRF2 has an amino acid sequence that is at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to the amino acid sequence set forth in GenBank/NCBI database accession no. NP_002190. In certain embodiments, the nucleic acid molecule encodes a human IRF2 that may contain substitutions (e.g., conservative substitutions), insertions, or deletions relative to the amino acid sequence set forth in GenBank/NCBI database accession no. NP_002190, that do not significantly alter the function or activity of the human IRF2.

In certain embodiments, the nucleic acid molecule encodes a mouse IRF2. In certain embodiments, the nucleic acid molecule encodes a mouse IRF2 having the amino acid sequence set forth in SEQ ID NO.: 2.

[SEQ ID NO: 2]
MPVERMRMRPWLEEQINSNTIPGLKWLNKEKKIFQIPWMHAARHGWDV
EKDAPLFRNWAIHTGKHQPGIDKPDPKTWKANFRCAMNSLPDIEEVKDR
SIKKGNNAFRVYRMLPLSERPSKKGKKPKTEKEERVKHIKQEPVESSLG
LSNGVSGFSPEYAVLTSAIKNEVDSTVNIIVVGQSHLDSNIEDQEIVTN
PPDICQVVEVTTESDDQPVSMSELYPLQISPVSSYAESETTDSVASDEE
NAEGRPHWRKRSIEGKQYLSNMGTRNTYLLPSMATFVTSNKPDLQVTIK
EDSCPMPYNSSWPPFTDLPLPAPVTPTPSSSRPDRETRASVIKKTSDIT
QARV

In certain embodiments, the mouse IRF2 has an amino acid sequence that is at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to the amino acid sequence set forth in GenBank/NCBI database accession no. NP_032417. In certain embodiments, the nucleic acid molecule encodes a mouse IRF2 that may contain substitutions (e.g., conservative substitutions), insertions, or deletions relative to the amino acid sequence set forth in GenBank/NCBI database accession no. NP_032417, that do not significantly alter the function or activity of the mouse IRF2.

In certain embodiments, conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. Amino acids can also be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. In certain embodiments, no more than one, no more than two, no more than three, no more than four, no more than five residues within a specified sequence are altered. Exemplary conservative amino acid substitutions are shown in Table 1 below.

TABLE 1

| | Exemplary Conservative Amino |
|---|---|
| Original Residue | Acid Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gin (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

5 As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Any suitable oncolytic viruses can be used with the presently disclosed subject matter. Non-limited examples of oncolytic viruses that can be used with the presently disclosed subject matter include Coxsackieviruses, Myxoma viruses, Maraba viruses (rhabdovirus), Parvoviruses, Seneca Valley viruses, vesicular stomatitis viruses (VSVs), Newcastle disease viruses (NDVs), retroviruses, reoviruses, measles viruses, Sindbis viruses, influenza viruses, herpes simplex viruses (HSVs), Sendai viruses, vaccinia viruses (VVs), and adenoviruses, and variants thereof.

In certain embodiments, the oncolytic virus disclosed herein is an oncolytic vaccinia virus. Any suitable strains of vaccinia viruses can be used with the presently disclosed subject matter. Non-limiting examples of vaccinia virus strains can be used with the presently disclosed subject matter include strains of, derived from, or modified forms of Western Reserve (WR) strain, Tashkent strain, Lister strain (also known as Elstree), Dryvax strain (also known as Wyeth strain), IHD-J strain, and IHD-W strain, Brighton strain, Ankara strain, modified vaccinia Ankara (MVA) strain, Dairen strain (e.g., Dairen I strain (DIs)), LIPV strain, lister clone 16m8 (LC16m8) strain, LC16MO strain, LIVP strain, WR 65-16 strain, Connaught strain, New York City Board of Health (NYCBH) strain, EM63 strain, ACAM2000™ strain, CV-1 strain, Paris strain, Copenhagen (Cop) strain, Bern strain, USSR strain, Evans strain, and the Tian Tan (VTT) strain.

Additional non-limiting examples of oncolytic viruses that can be used with the present disclosure include Talimogene Laherparepvec (T-Vec) (Amgen), TBI-1401 (HF10) (Takara), HSV1716 (Virtu Biologics), ADV/HSV-tk (Merk), LOAd703 (Loken), CG0070 (Cold Genesys), ColoAd1 (Enadenotucirev) (PsiOxus), ONCOS-102 (Targovax Oy), DNX-2401 (DNAtrix), VCN-01 (VCN), Ad-MAGEA3 and MG1-MAGEA3 (Turnstone), NSC-CRAd-Survivin-pk7 (Northwestern), Ad5-yCD/mutTKSR39rep-hIL 12 (Henry Ford), Ad5-yCD/mutTKSR39rep-ADP (Henry Ford), MV-NIS (Mayo), MV-NIS (University of Arkansas), GL-ONCI (Genelux), Pexastimogene Devacirepvec (Pexa-Vec) (Jennerx), REOLYSIN (Oncolytics), CVA21 (CAVATAK) (Viralytics), H-1PV (ParvOryx) (Oryx GmbH), PVSRIPO (Duke), vvDD (NIH), TBio-6517 (Turnstone), and VSV-hIFNbeta-NIS (Mayo).

In certain embodiments, the nucleic acid molecule encoding the IRF modulator is integrated into the genome of the oncolytic virus, where the expression of the nucleic acid molecule is operably linked to a promoter that is active or activatable in an oncolytic virus infected cell, for example, a promoter of the oncolytic virus. As used herein, “operably linked” means that a promoter is in a correct functional location and/or orientation in relation to a nucleic acid locus to control transcriptional initiation and/or expression of that locus.

In certain embodiments, the promoter is a vaccinia virus promoter. In certain embodiments, the vaccinia virus promoter is a synthetic vaccinia promoter. Non-limiting examples of vaccinia promoter can be used with the presently disclosed subject matter includes pSE/L and p7.5.

In certain embodiments, the oncolytic virus is attenuated to weaken viral pathogenicity and improve the safety of the therapeutic uses of the oncolytic virus. In certain embodiments, the oncolytic virus is a naturally attenuated strain. In certain embodiments, the oncolytic virus is genetically modified to weaken viral pathogenicity. In certain embodiments, the oncolytic vaccinia virus disclosed herein lacks the expression of a functional thymidine kinase (TK). In certain embodiments, the oncolytic vaccinia virus disclosed herein is TK negative. TK is encoded by the J2R gene (also known as tk gene), and forms part of the salvage pathway for pyrimidine deoxyribonucleotide synthesis. Lacking the expression of a functional TK can improve the safety of the oncolytic vaccinia virus. In certain embodiments, the oncolytic vaccinia virus includes a mutation of the J2R gene. In certain embodiments, the mutation of the J2R gene can be a deletion, a substitution, and/or an insertion of at least one nucleotide of the J2R gene nucleotide sequence. In certain embodiments, the mutation of the J2R gene includes an insertion of a nucleic acid molecule into the locus of the J2R gene.

In certain embodiments, a mutation in a gene (e.g., the J2R gene) is an inactivating mutation, in which the expression of the gene is significantly decreased, or the product encoded by the gene (e.g., TK) is rendered nonfunctional, or its ability to function is significantly decreased. In certain embodiments, the nucleic acid molecule encoding the IRF modulator (e.g., IRF2) is integrated into the J2R locus.

Additional approaches, beside modifying TK expression, can be used to create attenuated oncolytic viruses and improve the safety of the therapeutic uses of the oncolytic viruses. Non-limiting examples of attenuated oncolytic viruses include vSP virus (Guo et al., Cancer Res. 2005 Nov. 1; 65 (21): 9991-8), Modified vaccinia Ankara (MVA) (Harrop et al., Clin Cancer Res. 2006 Jun. 1; 12 (11 Pt 1): 3416-24), vvDD, a double viral gene-deleted (tk- and vgf-) vaccinia virus, as disclosed in McCart et al., Cancer Res 2001; 61:8751-7, and ACAM200 (Osborne et al., Vaccine. 2007 Dec. 17; 25 (52): 8807-32), the contents of which are incorporated herein by reference in their entireties.

### 5.3 Pharmaceutical Compositions

The present disclosure provides pharmaceutical compositions that include an oncolytic virus comprising a nucleic acid molecule that encodes an IRF modulator (e.g., an oncolytic virus disclosed in Section 5.2). In certain embodiments, the pharmaceutical compositions include an effective amount of the presently disclosed oncolytic virus.

In certain embodiments, the pharmaceutical compositions include an amount of the oncolytic virus of between about 10³ plaque forming units (PFU) and about 10¹³ PFU. In certain embodiments, the pharmaceutical compositions include an amount of the oncolytic virus of between about 10⁵ PFU and about 10¹³ PFU, between about 10⁵ PFU and about 10¹² PFU, between about 10⁵ PFU and about 10¹¹ PFU, between about 10⁵ PFU and about 10¹⁰ PFU, between about 10⁵ PFU and about 10⁹ PFU, between about 10⁵ PFU and about 10⁸ PFU, between about 10⁵ PFU and about 10⁷ PFU, between about 10⁵ PFU and about 10⁶ PFU, between about 10⁶ PFU and about 10¹³ PFU, between about 10⁶ PFU and about 10¹² PFU, between about 10⁶ PFU and about 10¹¹ PFU, between about 10⁶ PFU and about 10¹⁰ PFU, between about 10⁶ PFU and about 10⁹ PFU, between about 10⁶ PFU and about 10⁸ PFU, between about 10⁶ PFU and about 10⁷ PFU, between about 10⁷ PFU and about 10¹³ PFU, between about 10⁷ PFU and about 10¹² PFU, between about 10⁷ PFU and about 10¹¹ PFU, between about 10⁷ PFU and about 10¹⁰ PFU, between about 10⁷ PFU and about 10⁹ PFU, between about 10⁷ PFU and about 10⁸ PFU, between about 10⁸ PFU and about 10¹³ PFU, between about 10⁸ PFU and about 10¹² PFU, between about 10⁸ PFU and about 10¹¹ PFU, between about 10⁸ PFU and about 10¹⁰ PFU, between about 10⁸ PFU and about 10⁹ PFU, or between about 10⁹ PFU and about 10¹⁰ PFU. In certain embodiments, the pharmaceutical compositions include an amount of the oncolytic virus of at least about 1×10⁵ PFU, at least about 5×10⁵ PFU, at least about 1×10⁶ PFU, at least about 5×10⁶ PFU, at least about 1×10⁷ PFU, at least about 5×10⁷ PFU, at least about 1×10⁸ PFU, at least about 5×10⁸ PFU, at least about 1×10⁹ PFU, at least about 5×10⁹ PFU, at least about 1×10¹⁰ PFU, at least about 5×10¹⁰ PFU, at least about 1×10¹¹ PFU, at least about 5×10¹¹ PFU, at least about 1×10¹² PFU, at least about 5×10¹² PFU, or at least about 1×10¹³ PFU. In certain embodiments, the pharmaceutical compositions include an amount of the oncolytic virus of about 1×10⁵ PFU, about 5×10⁵ PFU, about 1×10⁶ PFU, about 5×10⁶ PFU, about 1×10⁷ PFU, about 5×10⁷ PFU, about 1×10⁸ PFU, about 5×10⁸ PFU, about 1×10⁹ PFU, about 5×10⁹ PFU, about 1×10¹⁰ PFU, about 5×10¹⁰ PFU, about 1×10¹¹ PFU, about 5×10¹¹ PFU, about 1×10¹² PFU, about 5×10¹² PFU, or about 1×10¹³ PFU. In certain embodiments, the pharmaceutical compositions include an amount of the oncolytic virus of between about 1×10⁶ PFU and about 3×10⁹ PFU. In certain embodiments, the pharmaceutical compositions include an amount of the oncolytic virus of between about 10⁸ PFU and about 10⁹ PFU, between about 10⁹ PFU and about 10¹⁰ PFU, or between about 10⁶ PFU and about 10⁷ PFU. In certain embodiments, the pharmaceutical compositions include an amount of the oncolytic virus of about 2.5×10⁶ PFU, about 1×10⁷ PFU, about 5×10⁸ PFU, about 6×10⁸ PFU, about 2×10⁹, about 2.5×10⁹, or about 3×10⁹ PFU.

In certain embodiments, the pharmaceutical compositions can be prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof.

In certain embodiments, the pharmaceutical compositions described herein further includes a pharmaceutically acceptable carrier, e.g., an excipient. In certain embodiments, the pharmaceutically acceptable carrier includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically acceptable carriers include gels, bioabsorbable matrix materials, implantation elements containing the oncolytic virus, and any other suitable vehicle, delivery, or dispensing means or material.

In certain embodiments, the pharmaceutically acceptable carrier can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide other calcium salts, and combinations thereof.

In certain embodiments, the oncolytic virus disclosed herein can be propagated in suitable host cells, isolated from host cells, and stored in conditions that promote stability and integrity of the virus, such that loss of infectivity over time is minimized. In certain embodiments, the oncolytic virus disclosed herein can be stored by freezing or drying, such as by lyophilization. In certain embodiments, prior to administration, the stored oncolytic virus can be reconstituted (if dried for storage) and diluted in a pharmaceutically acceptable carrier for administration.

In certain embodiments, the pharmaceutical compositions disclosed herein can further include an immunomodulatory agent (e.g., an immunomodulatory agent disclosed in Section 5.4).

In certain embodiments, the pharmaceutical compositions disclosed herein comprise an oncolytic virus comprising a nucleic acid molecule that encodes a modulator of an IRF (e.g., an oncolytic virus disclosed in Section 5.2) and a pharmaceutically acceptable carrier. In certain embodiments, the pharmaceutical compositions disclosed herein comprise an oncolytic virus comprising a nucleic acid molecule that encodes a modulator of an IRF (e.g., an oncolytic virus disclosed in Section 5.2) and an excipient and/or a buffering agent.

In certain embodiments, the pharmaceutical compositions disclosed herein comprise an oncolytic virus comprising a nucleic acid molecule that encodes an IRF modulator that inhibits the activity of IRF (e.g., IRF1) and a pharmaceutically acceptable carrier. In certain embodiments, the pharmaceutical compositions disclosed herein comprise an oncolytic virus comprising a nucleic acid molecule that encodes an IRF modulator that inhibits the activity of IRF (e.g., IRF1) and an excipient and/or a buffering agent.

In certain embodiments, the pharmaceutical compositions disclosed herein comprise an oncolytic virus comprising a nucleic acid molecule that encodes IRF2 and a pharmaceutically acceptable carrier. In certain embodiments, the pharmaceutical compositions disclosed herein comprise an oncolytic virus comprising a nucleic acid molecule that encodes IRF2 and an excipient and/or a buffering agent.

In certain embodiments, the pharmaceutical compositions disclosed herein comprise an oncolytic virus comprising a nucleic acid molecule that encodes a modulator of an IRF (e.g., an oncolytic virus disclosed in Section 5.2) and an immunomodulatory agent (e.g., an immunomodulatory agent disclosed in Section 5.4). In certain embodiments, the pharmaceutical compositions disclosed herein comprise an oncolytic virus comprising a nucleic acid molecule that encodes an IRF modulator that inhibits the activity of IRF (e.g., IRF1) and an immune checkpoint inhibitor. In certain embodiments, the pharmaceutical compositions disclosed herein comprise an oncolytic virus comprising a nucleic acid molecule that encodes IRF2 and an immune checkpoint inhibitor.

In certain embodiments, the pharmaceutical compositions disclosed herein comprise an oncolytic virus comprising a nucleic acid molecule that encodes IRF2 and an immune checkpoint inhibitor selected from the group consisting of anti-PD1 antibodies, anti-PD-L1 antibodies, anti-CTLA-4 antibodies, anti-BTLA antibodies, anti-TIM3 antibodies, anti-LAG-3 antibodies, and any combinations thereof. In certain embodiments, the pharmaceutical compositions disclosed herein comprise an oncolytic virus comprising a nucleic acid molecule that encodes IRF2 and an anti-PD-L1 antibody. In certain embodiments, the pharmaceutical compositions disclosed herein comprise an oncolytic virus comprising a nucleic acid molecule that encodes IRF2 and an anti-CTLA-4 antibody.

In certain embodiments, the pharmaceutical compositions disclosed herein comprise an oncolytic virus comprising a nucleic acid molecule that encodes a modulator of an IRF (e.g., an oncolytic virus disclosed in Section 5.2), an immunomodulatory agent (e.g., an immunomodulatory agent disclosed in Section 5.4), and a pharmaceutically acceptable carrier. In certain embodiments, the pharmaceutical compositions disclosed herein comprise an oncolytic virus comprising a nucleic acid molecule that encodes IRF2, an immune checkpoint inhibitor, and a pharmaceutically acceptable carrier. In certain embodiments, the pharmaceutical compositions disclosed herein comprise an oncolytic virus comprising a nucleic acid molecule that encodes IRF2, an anti-PD-L1 antibody or an anti-CTLA-4 antibody, and a pharmaceutically acceptable carrier.

### 5.4 Methods of Treatment

The present disclosure provides methods of treating a subject having cancer. In certain embodiments, the methods include administering to the subject an oncolytic virus comprising a nucleic acid molecule that encodes an IRF modulator (e.g., an oncolytic virus disclosed in Section 5.2) or a pharmaceutical composition comprising said oncolytic virus (e.g., a pharmaceutical composition disclosed in Section 5.3). In certain embodiments, the methods include administering to the subject an oncolytic virus comprising a nucleic acid molecule that encodes an IRF modulator that inhibits the activity of IRF (e.g., IRF1), or a pharmaceutical composition comprising said oncolytic virus. In certain embodiments, the methods include administering to the subject an oncolytic virus comprising a nucleic acid molecule that encodes IRF2, or a composition comprising said oncolytic virus.

In certain embodiments, the methods disclosed herein reduce aggregated cancer cell mass, reduce cancer cell growth rate, reduce cancer cell proliferation, reduce tumor mass, reduce tumor volume, reduce tumor weight, reduce tumor cell proliferation, reduce tumor growth rate, and/or reduce tumor metastasis in the subject.

Methods disclosed herein can be used for treating any suitable cancers. Non-limiting examples of cancers that can be treated by methods disclosed herein include adenocarcinomas, osteosarcomas, cervical carcinomas, melanomas, hepatocellular carcinomas, breast cancers, lung cancers, prostate cancers, ovarian cancers, leukemias, lymphomas, renal carcinomas, pancreatic cancers, gastric cancers, colon cancers, duodenal cancers, glioblastoma multiforme, astrocytomas, sarcomas, and combinations thereof.

In certain embodiments, methods disclosed herein can be used for treating solid tumors. In certain embodiments, methods disclosed herein can be used for treating melanomas. In certain embodiments, methods disclosed herein can be used for treating renal carcinomas.

In certain embodiments, the subject is a human subject. In certain embodiments, the subject is a non-human subject, such as, but not limited to, a non-primate, a dog, a cat, a horse, a rabbit, a mice, a rat, a guinea pig, a fowl, a cow, a goat or a sheep.

In certain embodiments, the methods disclosed herein include administering the oncolytic virus to the subject in an amount of between about 10³ and 10¹³ PFU. In certain embodiments, the methods disclosed herein includes administering the oncolytic virus to the subject in an amount of between about 10⁵ and 10¹³ PFU. In certain embodiments, the methods disclosed herein include administering the oncolytic virus to the subject in an amount of between about 10⁵ PFU and about 10¹³ PFU, between about 10⁵ PFU and about 10¹² PFU, between about 10⁵ PFU and about 10¹¹ PFU, between about 10⁵ PFU and about 10¹⁰ PFU, between about 10⁵ PFU and about 10⁹ PFU, between about 10⁵ PFU and about 10⁸ PFU, between about 10⁵ PFU and about 10⁷ PFU, between about 10⁵ PFU and about 10⁶ PFU, between about 10⁶ PFU and about 10¹³ PFU, between about 10⁶ PFU and about 10¹² PFU, between about 10⁶ PFU and about 10¹¹ PFU, between about 10⁶ PFU and about 10¹⁰ PFU, between about 10⁶ PFU and about 10⁹ PFU, between about 10⁶ PFU and about 10⁸ PFU, between about 10⁶ PFU and about 10⁷ PFU, between about 10⁷ PFU and about 10¹³ PFU, between about 10⁷ PFU and about 10¹² PFU, between about 10⁷ PFU and about 10¹¹ PFU, between about 10⁷ PFU and about 10¹⁰ PFU, between about 10⁷ PFU and about 10⁹ PFU, between about 10⁷ PFU and about 10⁸ PFU, between about 10⁸ PFU and about 10¹³ PFU, between about 10⁸ PFU and about 10¹² PFU, between about 10⁸ PFU and about 10¹¹ PFU, between about 10⁸ PFU and about 10¹⁰ PFU, between about 10⁸ PFU and about 10⁹ PFU. In certain embodiments, the methods disclosed herein include administering the oncolytic virus to the subject in an amount of at least about 1×10⁵ PFU, at least about 5× 10⁵ PFU, at least about 1×10⁶ PFU, at least about 5×10⁶ PFU, at least about 1×10⁷ PFU, at least about 5×10⁷ PFU, at least about 1×10⁸ PFU, at least about 5×10⁸ PFU, at least about 1×10⁹ PFU, at least about 5×10⁹ PFU, at least about 1×10¹⁰ PFU, at least about 5×10¹⁰ PFU, at least about 1×10¹¹ PFU, at least about 5×10¹¹ PFU, at least about 1×10¹² PFU, at least about 5×10¹² PFU, or at least about 1×10¹³ PFU. In certain embodiments, the methods disclosed herein include administering the oncolytic virus to the subject in an amount of about 1×10⁵ PFU, about 5×10⁵ PFU, about 1×10⁶ PFU, about 5×10⁶ PFU, about 1×10⁷ PFU, about 5×10⁷ PFU, about 1×10⁸ PFU, about 5×10⁸ PFU, about 1×10⁹ PFU, about 5×10⁹ PFU, about 1×10¹⁰ PFU, about 5×10¹⁰ PFU, about 1×10¹¹ PFU, about 5×10¹¹ PFU, about 1×10¹² PFU, about 5×10¹² PFU, or about 1×10¹³ PFU. In certain embodiments, the methods disclosed herein include administering the oncolytic virus to the subject in an amount of between about 1×10⁶ PFU and about 3×10⁹ PFU, between about 10⁸ PFU and about 10⁹ PFU, between about 10⁹ PFU and about 10¹⁰ PFU, or between about 10⁶ PFU and about 10⁷ PFU. In certain embodiments, the methods disclosed herein include administering the oncolytic virus to the subject in an amount of about 2.5×10⁶ PFU, about 1×10⁷ PFU, about 5×10⁸ PFU, about 6×10⁸ PFU, about 2×10⁹ PFU, about 2.5×10⁹ PFU, or about 3×10⁹ PFU.

In certain embodiments, the methods disclosed herein comprise administering to the subject the oncolytic virus in a single dose, or in multiple doses. In certain embodiments, where the oncolytic virus is administered to the subject in multiple doses, the doses can be administered sequentially, e.g., at daily, weekly, or monthly intervals, or in response to a specific need of the subject.

Any suitable methods of administration can be used with the presently disclosed subject matter for administering the oncolytic virus to the subject. In certain embodiments, the oncolytic virus disclosed herein is administered systemically. In certain embodiments, the oncolytic virus disclosed herein can be administered directly to a tumor site, e.g., via direct intratumoral injection.

For example, and not by way of limitation, the route of administration can be inhalation, intranasal, intravenous, intraarterial, intrathecal, intratumoral, intraperitoneal, intramuscular, subcutaneous, topical, intradermal, local regional, oral administration, or a combination thereof. In certain embodiments, the oncolytic virus disclosed herein is administered to the subject from a source implanted in the subject. In certain embodiments, the oncolytic virus disclosed herein is administered to the subject by continuous infusion over a selected period of time.

The present disclosure further provides methods for improving a subject's responsiveness to an immunomodulatory agent. In certain embodiments, the methods comprise administering to the subject an oncolytic virus comprising a nucleic acid molecule that encodes an IRF modulator (e.g., an oncolytic virus disclosed in Section 5.2) or a pharmaceutical composition comprising said oncolytic virus (e.g., a pharmaceutical composition disclosed in Section 5.3). In certain embodiments, the subject had previously been treated with the immunomodulatory agent. In certain embodiments, the subject has developed a resistance to the immunomodulatory agent. In certain embodiments, the methods further comprise administering the immunomodulatory agent to the subject in combination with the oncolytic virus disclosed herein.

The present disclosure also provides methods of treating a subject having a cancer, including administering to the subject an oncolytic virus comprising a nucleic acid molecule that encodes an IRF modulator (e.g., an oncolytic virus disclosed in Section 5.2) in combination with an immunomodulatory agent.

Any suitable immunomodulatory agent that targets components of the immune system to fight cancer can be used with the presently disclosed methods. Non-limiting examples of immunomodulatory agents include immune checkpoint inhibitors, T cells, dendritic cells, therapeutic antibodies (e.g., anti-CD33 antibodies, anti-CD11b antibodies), cancer vaccines, cytokines (e.g., IL-12, GM-CSF, IL-2, IFNβ, IFN-γ, MIP-1, MCP-1, IL-8), Bacillus Calmette-Guérin (BCG), and any combinations thereof. In certain embodiments, the immunomodulatory agent is an immune checkpoint inhibitor. In certain embodiments, the immune checkpoint inhibitor is selected from anti-PD1 antibodies, anti-PD-L1 antibodies, anti-CTLA-4 antibodies, anti-BTLA antibodies, anti-TIM3 antibodies, anti-LAG-3 antibodies, and any combinations thereof. Non-limiting examples of anti-PD1 antibodies include pembrolizumab (Keytruda), nivolumab (Opdivo), cemiplimab (Libtayo), and combinations thereof. Non-limiting examples of anti-PD-L1 antibodies include atezolizumab (Tecentriq), avelumab (Bavencio), durvalumab (Imfinzi), and combinations thereof. Non-limiting examples of anti-CTLA-4 antibodies include ipilimumab (Yervoy). In certain embodiments, the immunomodulatory agent is an anti-PD-L1 antibody. In certain embodiments, the immunomodulatory agent is an anti-CTLA-4 antibody.

In certain embodiments, the methods include administering to the subject an oncolytic virus comprising a nucleic acid molecule that encodes an IRF modulator that inhibits the activity of IRF (e.g., IRF1) in combination with an immunomodulatory agent. In certain embodiments, the methods include administering to the subject an oncolytic virus comprising a nucleic acid molecule that encodes an IRF modulator that inhibits the activity of IRF (e.g., IRF1) in combination with an immune checkpoint inhibitor. In certain embodiments, the methods include administering to the subject an oncolytic virus comprising a nucleic acid molecule that encodes an IRF modulator that inhibits the activity of IRF (e.g., IRF1) in combination with an anti-PD-L1 antibody or an anti-CTLA-4 antibody.

In certain embodiments, the methods include administering to the subject an oncolytic virus comprising a nucleic acid molecule that encodes IRF2 in combination with an immunomodulatory agent. In certain embodiments, the methods include administering to the subject an oncolytic virus comprising a nucleic acid molecule that encodes IRF2 in combination with an immune checkpoint inhibitor. In certain embodiments, the methods include administering to the subject an oncolytic virus comprising a nucleic acid molecule that encodes IRF2 in combination with an anti-PD-L1 antibody or an anti-CTLA-4 antibody. In certain embodiments, the oncolytic virus and the immunomodulatory agent can be administered to the subject as part of a treatment regimen. In certain embodiments, the oncolytic virus and the immunomodulatory agent can be administered concurrently to the subject. In certain embodiments, the oncolytic virus and the immunomodulatory agent can be administered at the same time. In certain embodiments, the oncolytic virus and the immunomodulatory agent can be administered sequentially in any order (e.g., the oncolytic virus is administered to the subject before the immunomodulatory agent is administered; or the oncolytic virus is administered to the subject after the immunomodulatory agent is administered) or at different points in time (e.g., the oncolytic virus and the immunomodulatory agent are administered to the subject on the same day but different hours; the oncolytic virus and the immunomodulatory agent are administered to the subject in the same week but on different days).

### 5.5 Kits

The present invention further provides kits that include an oncolytic virus comprising a nucleic acid molecule that encodes an IRF modulator (e.g., an oncolytic virus disclosed in Section 5.2) or a pharmaceutical composition comprising said oncolytic virus (e.g., a pharmaceutical composition disclosed in Section 5.3). In certain embodiments, the kits include an oncolytic virus comprising a nucleic acid molecule that encodes an IRF modulator that inhibits the activity of IRF (e.g., IRF1), or a pharmaceutical composition comprising said oncolytic virus. In certain embodiments, the kits include an oncolytic virus comprising a nucleic acid molecule that encodes IRF2, or a composition comprising said oncolytic virus.

In certain embodiments, the kits disclosed herein can further include instructions. In certain embodiments, the instructions include a description of the oncolytic virus, and optionally a description of other components included in the kit. In certain embodiments, the kits include instructions for treating a subject having cancer or improving a subject's responsiveness to an immunomodulatory agent. In certain embodiments, the instructions further include a description of methods for administration, including methods for determining the proper state of the subject, the proper dosage amount, and/or the proper administration method for administering the modified virus. In certain embodiments, the instructions further include guidance for monitoring the subject over duration of the treatment time.

In certain embodiments, the kits disclosed herein include a device for administering the oncolytic virus or the pharmaceutical composition to a subject. Any suitable devices known in the art for administering medications and pharmaceutical compositions can be included in the kits disclosed herein. For example, and not by way of limitation, suitable devices include, a hypodermic needle, an intravenous needle, a catheter, a needle-less injection device, an inhaler and a liquid dispenser, such as an eyedropper. In certain embodiments, an oncolytic virus to be delivered systemically, for example, by intravenous injection, can be included in a kit with a hypodermic needle and syringe.

In certain embodiments, the kits disclosed herein can further include an immunomodulatory agent (e.g., an immunomodulatory agent disclosed in Section 5.4). In certain embodiments, the immunomodulatory agent is an immune checkpoint inhibitor. In certain embodiments, the immune checkpoint inhibitor is selected from anti-PD1 antibodies, anti-PD-L1 antibodies, anti-CTLA-4 antibodies, anti-BTLA antibodies, anti-TIM3 antibodies, anti-LAG-3 antibodies, and any combinations thereof. In certain embodiments, the immunomodulatory agent is an anti-PD-L1 antibody. In certain embodiments, the immunomodulatory agent is an anti-CTLA-4 antibody.

In certain embodiments, the kits include an oncolytic virus comprising a nucleic acid molecule that encodes an IRF modulator (e.g., an oncolytic virus disclosed in Section 5.2) and an immune checkpoint inhibitor. In certain embodiments, the kits include an oncolytic virus comprising a nucleic acid molecule that encodes an IRF modulator that inhibits the activity of IRF (e.g., IRF1) and an immune checkpoint inhibitor. In certain embodiments, the kits include an oncolytic virus comprising a nucleic acid molecule that encodes IRF2 and an immune checkpoint inhibitor. In certain embodiments, the kits include an oncolytic virus comprising a nucleic acid molecule that encodes IRF2 and an anti-PD-L1 antibody or an anti-CTLA-4 antibody.

### 6. EXAMPLES

The presently disclosed subject matter will be better understood by reference to the following Example, which is provided as exemplary of the presently disclosed subject matter, and not by way of limitation.

### Example 1: Targeting IRFs: Targeted Expression of IRF2 Inhibited Tumor Growth

One factor that can affect a subject's responsiveness to immunotherapies is the interferon (IFN) response in tumor microenvironment. IFNs may play opposing roles in tumor cells as compared to immune cells. The presently disclosed subject matter utilizes this opposing IFN response by modulating molecules (e.g., IRFs) that regulate IFN response to improve the efficacy and responsiveness to immunotherapies. A number of CRISPR/Cas9-based gene-edited syngeneic tumor cells were created to establish that tumor intrinsic functions of specific IRFs, such as IRF1, IRF3 and IRF7, may underlie the opposing IFN response in tumor cells versus the host non-tumor immune cells. The present disclosure also discovered that targeting IRFs in tumor microenvironment using oncolytic viruses had therapeutic benefit. The present disclosure further developed IRF2-based transcriptional modulator to modulate IRF function in the tumor microenvironment using engineered oncolytic virus. The present disclosure discovered that IRF2-expressing oncolytic vaccinia virus successfully reduced tumor burden in preclinical mouse models.

IRF2 has been found to be deficient in primary human cancers, such as lung cancers, colon cancers, breast cancers, prostate cancers and others. The present disclosure discovered that IRF2 can inhibit IRF1-mediated gene induction (e.g., PD-L1), and can promote anti-tumor immune response.

### In Vitro Overexpression of IRF2 in Cancer Cells

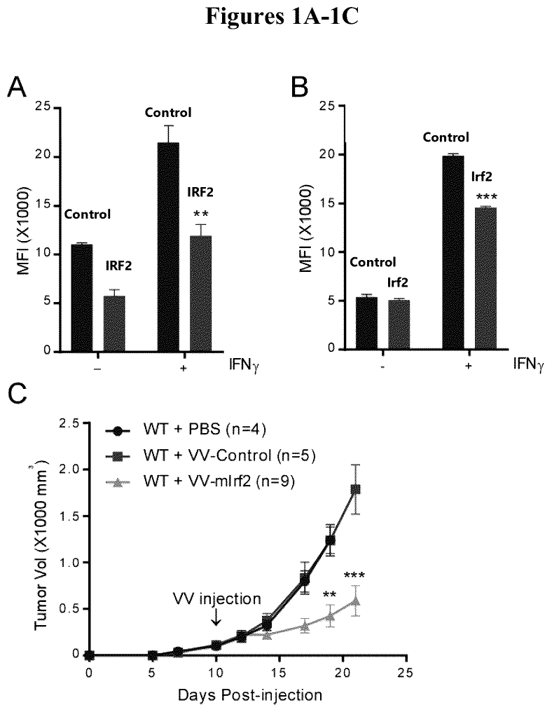

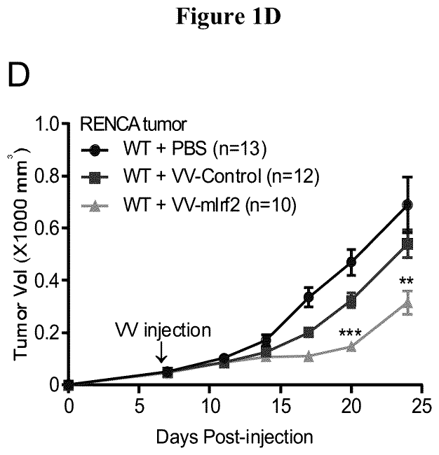

To evaluate whether IRF2 can modulate IRF1 activity and IRF1-mediated gene expression, IRF2 was overexpressed in human melanoma cells (MEL-285) and murine melanoma cells (B16). Viral vectors carrying human IRF2 gene or murine Irf2 gene were created. MEL-285 and B16 tumor cells were transfected with IRF2 carrying vectors or Irf2 carrying vectors respectively. The transfected cells were then stimulated with IFNγ. The expression of PD-L1 protein in MEL-285 and B16 tumor cells was evaluated by flow cytometry. Overexpression of IRF2 in human MEL-285 and murine B16 melanoma cells reduced the expression of PD-L1 in both cell lines (FIGS. 1A-1B).

In Vivo Administration of mIrf2-Expressing Oncolytic Vaccinia Virus in Preclinical Mouse Models

IRF-2 expressing oncolytic vaccinia viruses were created by inserting mouse Irf2 gene into the TK locus of the viral genome of oncolytic vaccinia viruses. The insertion disrupted the TK gene. In vivo studies were conducted to examine the anti-tumor activity of the mIrf2-expressing oncolytic viruses in two different mouse tumor models. Mice were implanted with B16 tumor cells (melanoma tumor cells) on day 0. Ten days after the implantation, the tumor bearing mice were intratumorally injected with PBS, 2.5×10⁶ PFU thymidine kinase deficient (TK-) vaccinia virus (VV-control), or 2.5×10⁶ PFU mIrf2-expressing oncolytic vaccinia viruses (VV-mIrf2). Tumor volume was monitored and measured for 22 days. Intratumoral injection of the mIrf2-expressing oncolytic vaccinia viruses significantly inhibited the growth of B16 tumor as compared to PBS and VV-controls (FIG. 1C).

The anti-tumor activity of the mIrf2-expressing oncolytic virus was further evaluated in a preclinical RENCA tumor (renal carcinoma) mouse model. RENCA tumor was established in BALB/C mice through subcutaneous injection. The tumor-bearing mice were intratumorally injected with PBS, 1×10⁷ PFU thymidine kinase deficient (TK-) vaccinia virus (VV-control), or 1×10⁷ PFU mIrf2-expressing oncolytic vaccinia viruses (VV-mIrf2). Tumor growth was monitored and measured. Intratumoral injection of VV-mIrf2 significantly inhibited the growth of RENCA tumors as compared to PBS control (FIG. 1D). Additionally, the anti-tumor effects of VV-control were significantly improved by mIrf-2 expression.

PD-L1/PD-1 axis is an essential immune checkpoints that can be exploited by cancer cells for evading immune detection and elimination. Efforts have been made to block immune checkpoint proteins including PD-L1 and PD-1, in order to overcome cancer's ability to evade the immune responses, and to stimulate host immune response in defending against cancer. The present disclosure demonstrated that IRF2 can effectively downregulate the expression of PD-L1 protein in cancer cells, and therefore inhibit the activation of PD-L1/PD-1 pathway. The present disclosure further suggested that overexpressing IRF2 in cancer cells can improve host immune response in attacking cancer cells, and may improve cancer cells' responsiveness to immunotherapies, such as immune checkpoint inhibitors (e.g., anti-PD-L1 antibodies).

Oncolytic viruses can selectively infect and lyse tumor cells, and can induce anti-tumor immune responses. The present disclosure demonstrated that incorporating an immunomodulatory gene IRF2 into the genome of the oncolytic virus significantly improved the anti-tumor activity of the oncolytic virus. These results demonstrated that IRF proteins had versatile functions in tumor microenvironment.

Although the presently disclosed subject matter and certain of its advantages have been described in detail, it should be understood that various changes, substitutions and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which are incorporated herein by reference in their entireties for all purposes.

What is claimed is:

1. An oncolytic virus comprising a nucleic acid molecule that encodes interferon regulatory factor 2 (IRF2), wherein the oncolytic virus lacks the expression of a functional thymidine kinase (TK).

2. The composition of claim 1, wherein the nucleic acid molecule encoding IRF2 is integrated into a J2R locus of the oncolytic virus genome.

3. The oncolytic virus of claim 1, wherein the IRF2 inhibits the activity of IRF1.

4. The oncolytic virus of claim 1, wherein the IRF2 reduces IRF-mediated gene expression.

5. The oncolytic virus of claim 1, wherein the IRF2 reduces the expression of CD274 gene.

6. The oncolytic virus of claim 1, wherein the IRF2 is a human IRF2 or a mouse IRF2.

7. The oncolytic virus of claim 1, wherein the nucleic acid molecule is an exogenous nucleic acid molecule.

8. The oncolytic virus of claim 1, wherein the nucleic acid molecule is integrated into the genome of the oncolytic virus.

9. The oncolytic virus of claim 1, wherein the oncolytic virus is an oncolytic vaccinia virus.

10. A method of treating a subject having cancer, comprising administering to the subject an oncolytic virus comprising a nucleic acid molecule that encodes interferon regulatory factor 2 (IRF2), wherein the oncolytic virus lacks the expression of a functional thymidine kinase (TK).

11. The method of claim 10, wherein the subject is a human subject.

12. The method of claim 10, further comprising administering an immunomodulatory agent to the subject.

13. The method of claim 12, wherein the immunomodulatory agent is selected from the group consisting of immune checkpoint inhibitors, T cells, dendritic cells, therapeutic antibodies, cancer vaccines, cytokines, Bacillus Calmette-Guérin (BCG), and any combinations thereof.

14. The method of claim 13, wherein the immunomodulatory agent is an immune checkpoint inhibitor.

15. The method of claim 13, wherein the immune checkpoint inhibitor is selected from the group consisting of anti-PD1 antibodies, anti-PD-L1 antibodies, anti-CTLA-4 antibodies, anti-BTLA antibodies, anti-TIM3 antibodies, anti-LAG-3 antibodies, and any combinations thereof.

16. The method of claim 13, wherein the immune checkpoint inhibitor is an anti-PD-L1 antibody or an anti-CTLA-4 antibody.

17. The method of any one of claim 10, wherein the cancer is a solid tumor.

18. The method of any one of claim 10, wherein the cancer is selected from the group consisting of adenocarcinomas, osteosarcomas, cervical carcinomas, melanomas, hepatocellular carcinomas, breast cancers, lung cancers, prostate cancers, ovarian cancers, leukemias, lymphomas, renal carcinomas, pancreatic cancers, gastric cancers, colon cancers, duodenal cancers, glioblastoma multiforme, astrocytomas, sarcomas, and combinations thereof.


SEQUENCE LISTING (2 sequences)

SEQ ID NO: 1 - protein, 349 aa, Homo sapiens
MPVERMRMRPWLEEQINSNTIPGLKWLNKEKKIFQIPWMHAARHGWDVEKDAPLFRNWAIHTGKHQPGVDKPDPKTWKANFRCAMNSLPDIEEVKDKSIKKGNNAFRVYRMLPLSERPSKKGKKPKTEKEDKVKHIKQEPVESSLGLSNGVSDLSPEYAVLTSTIKNEVDSTVNIIVVGQSHLDSNIENQEIVTNPPDICQVVEVTTESDEQPVSMSELYPLQISPVSSYAESETTDSVPSDEESAEGRPHWRKRNIEGKQYLSNMGTRGSYLLPGMASFVTSNKPDLQVTIKEESNPVPYNSSWPPFQDLPLSSSMTPASSSSRPDRETRASVIKKTSDITQARVKSC

SEQ ID NO: 2 - protein, 346 aa, Homo sapiens
MPVERMRMRPWLEEQINSNTIPGLKWLNKEKKIFQIPWMHAARHGWDVEKDAPLFRNWAIHTGKHQPGIDKPDPKTWKANFRCAMNSLPDIEEVKDRSIKKGNNAFRVYRMLPLSERPSKKGKKPKTEKEERVKHIKQEPVESSLGLSNGVSGFSPEYAVLTSAIKNEVDSTVNIIVVGQSHLDSNIEDQEIVTNPPDICQVVEVTTESDDQPVSMSELYPLQISPVSSYAESETTDSVASDEENAEGRPHWRKRSIEGKQYLSNMGTRNTYLLPSMATFVTSNKPDLQVTIKEDSCPMPYNSSWPPFTDLPLPAPVTPTPSSSRPDRETRASVIKKTSDITQARV